(19) United States
(12) Patent Application Publication
(10) Pub. No.: US 2023/0050412 A1
(43) Pub. Date: Feb. 16, 2023

(54) MEDICAL DIAGNOSTIC DEVICE

(71) Applicant: BILKENT UNIVERSITESI (UNAM) ULUSAL NANOTEKNOLOJI ARASTIRMA MERKEZI, Ankara (TR)

(72) Inventors: Urartu Ozgur Safak SEKER, Ankara (TR); Sila KOSE, Ankara (TR); Recep Erdem AHAN, Ankara (TR)

(73) Assignee: BILKENT UNIVERSITESI (UNAM) ULUSAL NANOTEKNOLOJI ARASTIRMA MERKEZI, Ankara (TR)

(21) Appl. No.: 17/789,866

(22) PCT Filed: Dec. 28, 2020
(86) PCT No.: PCT/TR2020/051415

§ 371 Date: Jun. 29, 2022

(30) Foreign Application Priority Data
Dec. 30, 2019 (TR) 2019/22560

(51) Int. Cl. G01N 33/50; G01N 33/62

(52) U.S. Cl. CPC G01N 33/5008; G01N 33/62

(57) ABSTRACT

The present invention relates to a medical diagnostic device with a cellular biosensor which detects urea and uric acid by means of a synthetic genetic circuit essentially consisting of transcriptional regulator and bio-sensing module.

### TECHNICAL FIELD

The present invention relates to a medical diagnostic device with a cellular biosensor which detects urea and uric acid by means of a synthetic genetic circuit essentially consisting of transcriptional regulator and bio-sensing module.

### BACKGROUND OF THE INVENTION

Urea is the end product of protein metabolism and it is excreted by kidneys in the urine. Increase of urea concentration in blood may be an indicator of renal dysfunctions. Therefore, urea acts as an important biomarker for monitoring kidney functions and detecting kidney-related diseases. In addition, urea concentration sensors are an important part of therapeutics developed in order to maintain urea homeostasis in patients with renal dysfunction. Besides clinical and therapeutic applications, measurements of urea amount are utilized in the food industry, particularly during production and quality control of dairy products. Additionally, since urea is used as a fertilizer in soil, it has a potential to contaminate water resources by causing formation of ammonia which is very toxic when decomposed. Therefore, urea biosensors also have a field of application in the environmental industry as well.

Numerous techniques have been developed for determination of urea. Most of the methods developed for determination of urea have an enzyme urease which hydrolyzes urea as a detection module. Methods of monitoring this enzymatic activity uses UV-visible spectrophotometry, potentiometry with pH electrode application, ammonium ion-selective electrode and ammonium ion-selective field effect transistor, carometry and amperometre. In addition, the most common technique for monitoring urease activity is potentiometric transducer which monitors potential change resulting from generation of ammonium or hydrogencarbonate ions with an ion-selective electrode. Yielding relative results in biological samples, being not suitable for conversion into portable measurement systems, having slow and low sensitivity are disadvantages of this technique.

Uric acid is a waste product of purine metabolism and it can he excreted both from the kidneys and the intestinal tract. An imbalance occurring in uric acid homeostasis in the body may trigger various pathologies such as gout, kidney diseases, tumor lysis syndrome and Lesch-Nyhan syndrome. Also, it is associated with conditions such as hyperuricemia, hypertension, metabolic syndromes and cardiovascular disease. In addition to this, uric acid levels are monitored for quality control in food products such as milk too. As a result, levels of uric acid are regularly controlled in clinical diagnostic studies because it is an important biomarker of various diseases.

There are two techniques commonly used for measurement of uric acid in hospitals. The first of these is a calorimetric method, and sodium tungstate and similar chromogens are used. Chromogen reduction with uric acid leads to a measurable colour change. Although this method is used in medical tests, due to the fact that results are affected by presence of ascorbic acid and similar molecules, it is generally accepted that results are measured above actual uric acid levels. The other most common method quantifies uric acid by means of an enzymatic method and uses uricase in order to convert uric acid into allantoin. This method measures differential absorbance in order to measure uric acid concentration. However, although it is more specific than a calorimetric method, it is more expensive.

Another disadvantage of the state of the art can be stated in such a way that, because natural promoters and transcription factors are optimized according to their natural environment, their response to inducers and working efficiency vary contextually and their adaptations becomes difficult. The fact that some cellular biosensors designed do not behave as expected may limit them to become improved and commercialized. Besides, it is observed that some improved whole-cell biosensors yield relative results in complex and heterogeneous samples and have low signal-to-noise ratio. Although there have been many improvements in cell-based biosensor systems, there is a limited improvement which is improved for providing more precise and personalized diagnosis and focuses on logic gate systems by combining a plurality of biomarkers. Therefore, there is need for a device which enables to differentiate diseases with overlapping biomarkers and yield precise results.

In the medical diagnostic, food and environmental industries, conventional technologies continually confront difficulties due to economic and resource constraints. For this reason, there is need for a device for performing diagnosis of urea and uric acid concentration so as to have low cost, fast scanning, high specificity and efficiency characteristics in the clinical, food and environmental industries.

The International patent document no. WO2012074358, an application in the state of the art, discloses a hybrid solgel modified electrode for detecting analytes in body fluids. A hybrid solgel modified electrode is a non-enzymatic biosensor. The said biosensor targets detection of biomolecules which include glucose, cholesterol, uric acid, creatine, urea and lactate. A hybrid solgel modified electrode comprises a substrate layer, a conductor layer and a hybrid solgel composite layer. The substrate layer, in the form of a bowl, is to provide mechanical strength to the electrode structure and also to give a good adhesion to the screen printed on the conductor layer. The conductor layer is a screen printed conducting layer to provide electrical contact between the hybrid solgel modified electrode and the readout circuitry. To cover the conductor and substrate layer, the hybrid solgel composite layer detects body analytes by selective voltammetric oxidation of the bio-molecules.

In the United States patent document no. US20040072369, another application in the state of the art, urea binding dynamics of the UreR protein are examined by creating a fusion protein with UreR and a fluorescent label. In the method disclosed, the protein bound to the urea is used for determining fluorescence change in a suitable fluorescent label environment conjugated to UreR. Here, the sulthydryl in the system at position 59 of the amino acid of UreR is used for covalently linking solvent-sensitive fluorophore NBD iodacetamide. As another way for monitoring urea concentration, fluorescent-labeled oligonucleotide and UreR are used at a suitable concentration within a urea-permeable chamber with DNA molecules, the UreR binding site. Then, fluorescence anisotropy can be measured from the chamber as an index of the movement of labeled DNA. Alternatively, competition of fluorescent thiourea and UreR protein for a binding site can be followed. In this case, anisotropin expected to decrease due, to the fact that a smaller fraction of the fluorescent thiourea will be in the urea binding site, at high urea concentrations. In another method whereby urea is detected in vitro and in situ, a plasmid is created by cloning UreR-intergenic Region-UreD′ fragment from P. mirabilis organism before GFP gene. This plasmid is then P. Mirabilis transformed and a mouse infection model is examined by this P. Mirabilis with sensor function.

In the International patent document no. WO2010133298, an application in the state of the art, a synthetic device is developed by using a HucR derived protein in D. Radiodurans that is able to detect uric acid for providing dose-dependent derepression of uric acid levels in the blood. Here, the repressor protein HucR is modified for optimum performance in mammalian cells. Changing the start codon, fusion by a Kozak consensus sequence and fusion of a domain of a Krueppel-associated box (KRAB) protein to a C terminal of the protein are among the changes made. As a promoter site, a simian virus 40 promoter is used for regulating expression of the urate oxidase enzyme converted into uric acid allantoine by eight tandem hucO module cloned thereafter.

### SUMMARY OF THE INVENTION

An Objective of the present invention is to realize a medical diagnostic device with a cellular biosensor which detects urea and uric acid by means of a synthetic genetic circuit essentially consisting of transcriptional regulator and bio-sensing module.

### DETAILED DESCRIPTION OF THE INVENTION

“A Medical Diagnostic Device” realized to fulfil the objective of the present invention is shown in the figures attached, in which:

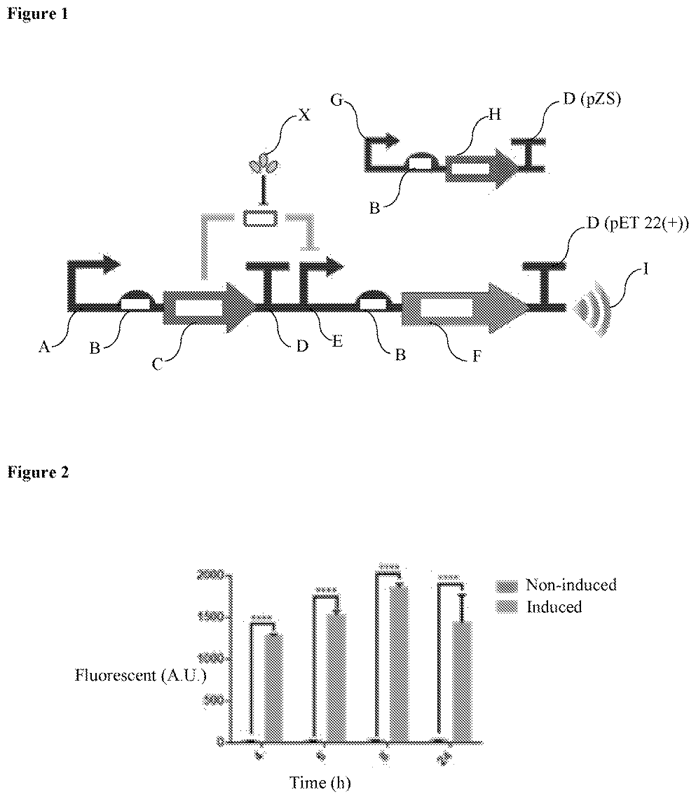

FIG. 1 is a schematic view of a cellular uric acid biosensor.

FIG. 2 is a chart for time-dependent fluorescent signal variation of a cellular uric acid biosensor.

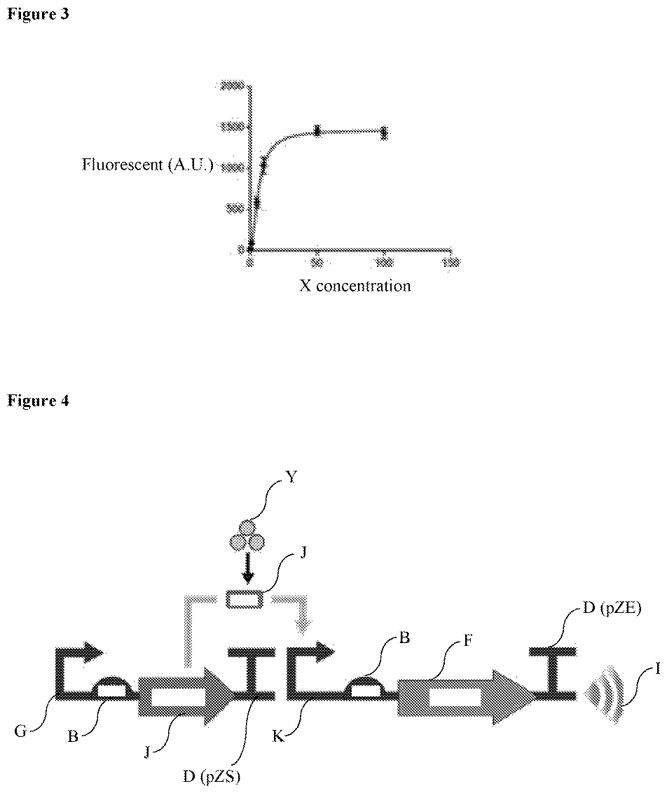

FIG. 3 is a chart for concentration-dependent characterization of a cellular uric acid biosensor at 8 hours after induction.

FIG. 4 is a schematic view of a cellular urea biosensor.

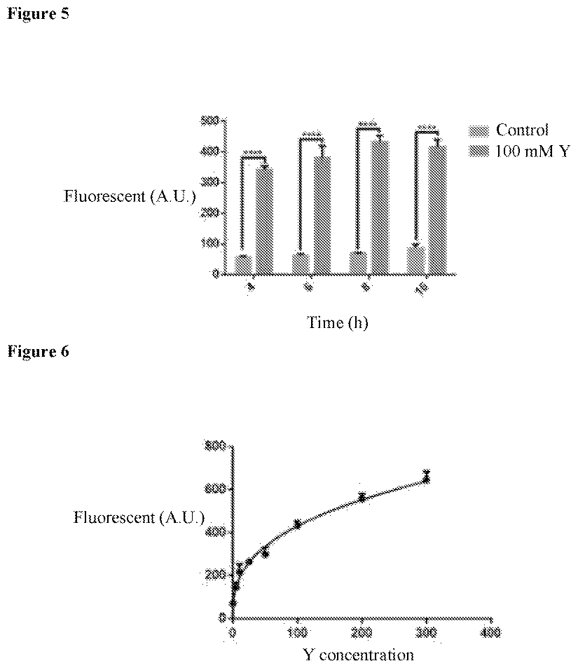

FIG. 5 is a chart for time-dependent fluorescent signal variation of a cellular urea biosensor.

FIG. 6 is a chart for concentration-dependent characterization of a cellular urea biosensor at 8 hours after induction.

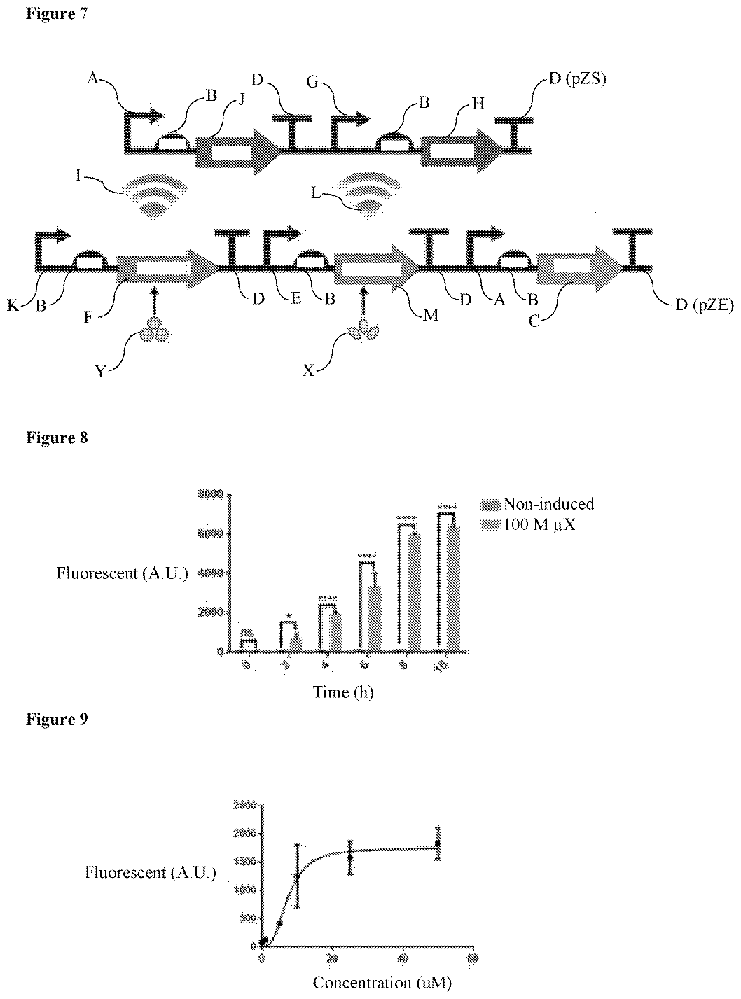

FIG. 7 is a schematic view of a biosensor with separate reporter for cellular urea and uric acid.

FIG. 8 is a chart for time-dependent red fluorescent signal variation of a biosensor with separate reporter for cellular urea and uric acid.

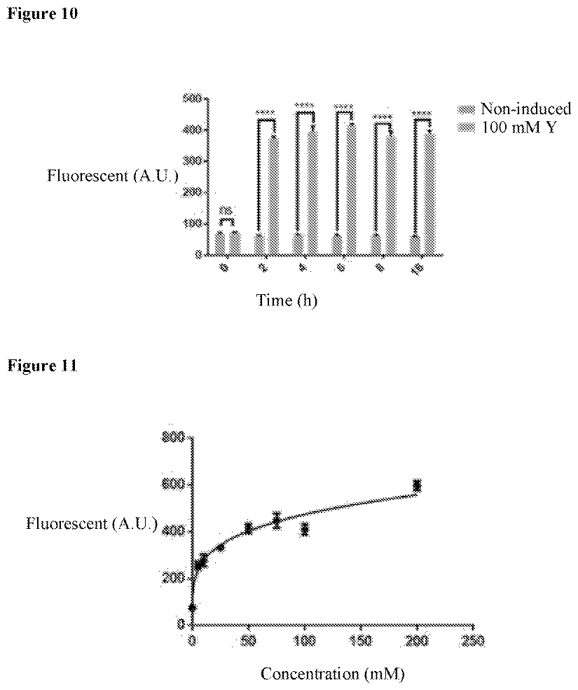

FIG. 9 is a chart for uric acid concentration-dependent characterization of a biosensor with separate reporter for cellular urea and uric acid at 8 hours after induction.

FIG. 10 is a chart for time-dependent green fluorescent signal variation of a biosensor with separate reporter for cellular urea and uric acid.

FIG. 11 is a chart for urea concentration-dependent characterization of a biosensor with separate reporter for cellular urea and uric acid at 8 hours after induction.

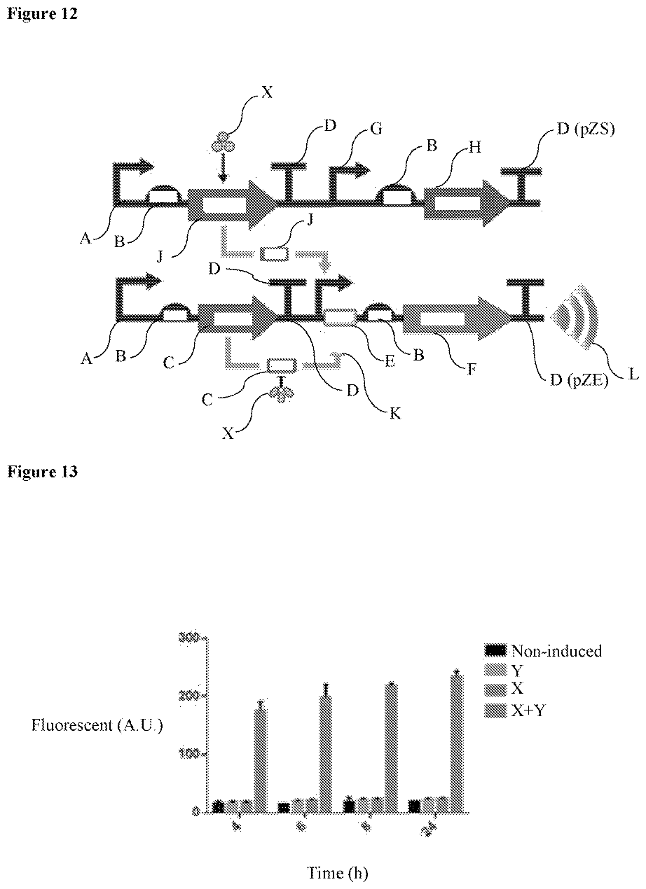

FIG. 12 is a schematic view of a urea and uric acid biosensor with AND-Logic gate.

FIG. 13 is a chart for time-dependent fluorescent signal variation of a urea and uric acid biosensor with AND-Logic gate.

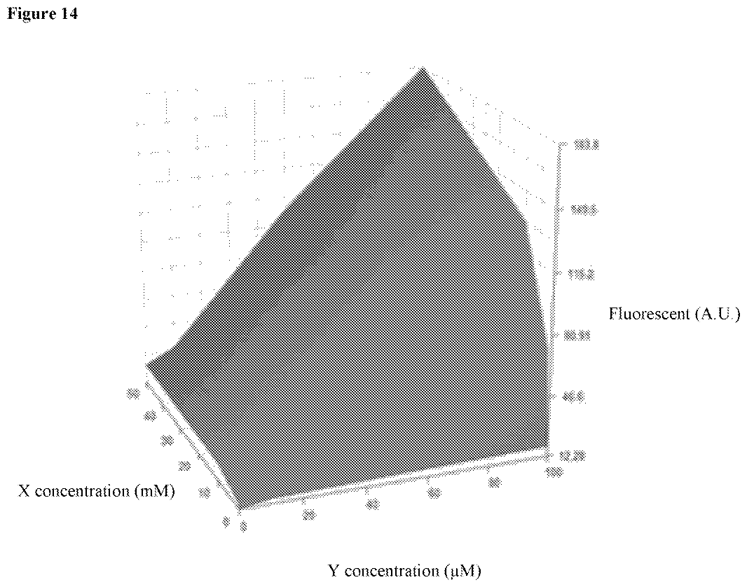

FIG. 14 is a chart for urea and uric acid concentration-dependent characterization of a urea and uric acid biosensor with AND-Logic, gate at 8 hours after induction.

The inventive medical diagnostic device comprises cellular biosensors performing diagnosis of urea and uric acid; and it is used for diagnosis of various diseases such as routine blood analysis and monitoring biomarkers about kidney health in the medical field. The said medical diagnostic device has capability of performing low-cost, quick scanning and characteristic of providing high-specificity and yield, by means of cellular sensors it contains.

The inventive medical diagnostic device is configured to comprise cellular biosensors that can perform urea detection, uric acid detection, urea and uric acid detection separately, and can detect that urea and uric acid are present in one environment at the same time. The biosensors included in the medical diagnostic device comprise synthetic genetic circuits. The said synthetic genetic circuits are essentially configured to have transcriptional regulator and bio-sensing module that vary by the component aimed to be detected. Cellular biosensors ensures formation of fluorescent signal increase in case of detecting urea and/or uric acid in the environment, and enables to detect this signal increase by fluorescence spectroscopy.

In one embodiment of the invention, the uric acid (X) used for detecting uric acid by the medical diagnostic device is cloned from uricase operator system in transcriptional regulator HucR (C) and its DNA binding site HucO (E), organism Deinococcus radiodurans for cellular biosensor parts. HucR (C) controls transcription negatively. DNA binding affinity of HucR (C) decreases by presence of uric acid in the environment and transcription occurs. The genetic circuit created for expression of HucR (C) protein consists of continuously active promoter site proD (A), ribosome binding site (RBS) (B), gene of HucR (C) protein and rrnB T1 transcriptional terminator (13) site. Here, the promoter region is responsible for initiating transcription upon the RNA polymerase binds Onto the plasmid whereas the rrnB T1 transcriptional terminator region (D) is used for terminating the RNA synthesis. The messenger RNA (mRNA) generated after the transcription initiates the translation by binding to the ribosome with the ribosome binding site placed before thereof, and ensures expression of the desired gene as protein. In this system, HucR (C) is generated by promotor proD (A) continuously. Synthetic promoter synpHucO (E) consists of HucO (E) operator placed between −35, −10 regions of viral promoter pL. Synthetic promoter and RBS (B) are cloned before reporter green fluorescent protein (sfGFP) in (F), and after T7 terminator site (D). Synthetic promoter synpHucO (E) is activated depending on the presence of uric acid because it contains HucO binding site. HucR (C) and sfGFP (F) expression modules are placed to pET22b (+) high copy plasmid. Entry of uric acid into the cell is possible by uric acid transporter (UACT) (H) gene cloned between minimal promotor mproD (G) that is continuously active on low copy plasmid pZS and RBS site (B) and T7 terminator (D) site. Lastly, cellular uric acid biosensor generates a GFP signal (I) in case of detecting uric acid in the environment. Schema, charts of time and concentration-dependent fluorescent expression profile of the uric acid biosensor are indicated in the FIGS. 1, 2 and 3 respectively.

In the FIG. 1, the transcriptional suppressor HucR is continuously expressed by promoter prod. The HucR prevents transcription from the synpHucO promoter by binding in the HucO binding site in the absence of uric acid. The SynpHucO promoter is placed before the reporter protein sfGFP. Both transcription factor and promoter-reporter gene circuits are cloned into high copy plasmid pET22b (+). In low copy plasmid pZS, UACT is expressed by low power mproD promoter. When uric acid is added to the system, HucR—which is transferred to the intracellular environment with UACT and binds to the uric acid—is prevented from binding to synpHucO. Thereby, the transcription carried out from the synpHucO does not lead to increase of fluorescent signal.

The FIG. 2 includes a chart for time-dependent fluorescent signal variation of a cellular uric acid biosensor. The cellular uric acid biosensor is induced by 50 μM uric acid solution.

The FIG. 3 includes a chart for concentration-dependent characterization of a cellular uric acid biosensor at 8 hours after induction. Experiments are carried out at 37′ C., 200 rpm in LB-liquid medium.

In another embodiment of the invention, the urea (Y) used for diagnosing urea with the medical diagnostic device is cloned from transcriptional regulator UreR (J) from cellular biosensor parts and urease specific promoter region (Intergenic Region) (K) used as a promoter that is inducible by urea, urease operon system of Proteus mirabilis organism. Production of UreR (J) in cells is ensured by cloning ureR (J) gene between mproD promotor (G) and RBS (B) that are continuously active on low copy plasmid pZS and T7 terminator (D). The ureR (J) has characteristics of binding to operator regions on the urease specific promoter region (K) and activating the transcription, when urea is present in the environment. The urease specific promoter region (K), the RBS (B), the reporter protein sfGFP (F) and the rrnb T1 terminator (D) are placed onto the high copy plasmid pZE. Thereby, the amount of urea in the environment can he detected by measuring the fluorescent signal (GFP signal (I)). Schema, charts of time and concentration-dependent fluorescent expression profile of the urea biosensors are indicated in the FIG. 4, 5 and respectively.

In the schematic view of the urea biosensor in the FIG. 4, the transcriptional regulator UreR generated by the promoter mproD is cloned to low copy plasmid pZS. In the absence of urea, the UreR cannot activate the transcription because it cannot bind onto the urease specific promoter region. The promoter of the urease specific promoter region is cloned before the reporter protein sfGFP and the promoter-reporter gene circuit is conveyed onto the high copy plasmid pZE. When urea is added into the system, the urea enters the cell by itself and activates the protein by binding to the UreR. A transcription performed over the maw specific promoter region leads to increase of fluorescent signal.

The FIG. 5 includes a chart for time-dependent fluorescent signal variation of a cellular urea biosensor. The cellular urea biosensor is induced by 100 mM urea solution.

The FIG. 6 includes a chart for concentration-dependent characterization of a cellular urea biosensor at 8 hours after induction.

In another embodiment of the invention, a system consisting of all components of independent urea and uric acid detection and processing modules for a cellular biosensor with separate reporter of urea and uric acid—that is used for diagnosing urea (V) and uric acid (X) by the medical diagnostic device—is designed. Genetic circuits of mproD (G)-RBS (B)-ureR (J)-T7 terminator (D) are used for expression of UreR (J) protein respectively and mproD (G)-RBS (B)-UACT (H)-rrnb T1 terminator (D) are used for expression of UACT (H) transporter, on the pZS plasmid respectively. Whereas genetic circuit of prop (A)-RBS (B)-HucR (C)-T7 terminator is cloned onto pZE plasmid for production of HucR (C) protein. The bin-recognition module on the bio-recognition module is designed such that it will generate sfGFP (F) protein as the reporter; whereas the (pUreD-RBS-sfGFP-rrnb T1 terminator), uric acid bio-recognition module is designed such that it will generate mScarlet I (M) reporter protein as the reporter protein (syn pHucO-RBS-mScarlet I- rrnb T1 terminator). Lastly, a GEP signal (green signal) (I) is generated when it is detected that there is urea in the cellular biosensor environment with separate promoter of the urea and the uric acid; and a RFP signal (red signal) (L) is generated when it is detected that there is uric acid. Schema, charts of time and concentration-dependent fluorescent expression profile of the biosensor with separate reporter for urea and uric acid are indicated in the FIG. 7, 8, 9, 10 and 11.

The FIG. 7 includes schematic view of a biosensor with separate reporter for urea and uric acid.

The FIG. 8 includes a chart for time-dependent red fluorescent signal variation of a biosensor with separate reporter for urea and uric acid. The biosensor is induced by 50 μM uric acid solution.

The FIG. 9 includes a chart for uric acid concentration-dependent characterization of a biosensor with separate reporter for urea and uric acid at 8 hours after induction.

The FIG. 10 includes a chart for time-dependent green fluorescent signal variation of a biosensor with separate reporter for urea and uric acid. The biosensor is induced by 100 mM urea solution.

The FIG. 11 includes a chart for urea concentration-dependent characterization of a biosensor with separate reporter for urea and uric acid at 8 hours after induction. Experiments are carried out at 37′ C., 200 rpm in LB-liquid medium.

In another embodiment of the invention, a synthetic promoter which is active only in the presence of urea and uric acid is used in the urea and uric acid biosensor with AND-Logic gate used for diagnosing that urea (Y) and uric acid (X) are present in the environment at the same time by the medical diagnostic device. Cloning a HucO (E) binding site between −35 and −10 regions of a promoter on the urease specific promoter region (K) makes the transcription activation dependent on the presence of both urea and uric acid. The synthetic AND-Logic gate promoter is cloned before the RBS (B), reporter sfGFP gene (F) and rrnB T1 terminator (D) region. The generated genetic circuit is combined in a single cell by components of other singular urea and uric acid detection, processing modules Genetic circuits of mproD (G)-RBS (B)-ureR (J)-T7 terminator (D) are used for expression of UreR (J) protein respectively and mproD (G)-RBS (B)-UACT (H)-rrnb T1 terminator (D) are used for expression of UACT (H) transporter, on the pZS plasmid respectively. Genetic circuit of proD (A)-RBS (B)-HucR (C)-T7 terminator (D) is cloned onto pZE plasmid for production of HucR (C) protein. Lastly, a GFP signal (I) is generated when it is detected that urea and uric acid are present at the same time in the urea and uric acid biosensor environment with AND-Logic gate. Schema, charts of time and concentration-dependent fluorescent expression profile of the biosensor with AND-Logic gate are indicated in the FIG. 12, 13, 14.

The FIG. 12 includes a schematic view of a urea and uric acid biosensor with AND-Logic gate

The FIG. 13 includes a chart for time-dependent fluorescent signal variation of a urea and uric acid biosensor with AND-Logic gate. The biosensor is induced by 50 μM uric acid solution and/or 100 mM urea solution.

The FIG. 14 includes a chart for urea and uric acid concentration-dependent characterization of a urea and uric acid biosensor with AND-Logic gate at 8 hours after induction. Experiments are carried out at 37° C. and 200 rpm in LB-liquid medium.

The cell-based biosensor technology included in the inventive medical diagnostic device is developed as a low-cost, quick and user-friendly diagnostic method for measurement in micro environments. Thereby, it can be used for frequent or real-time measurements of urea and uric acid. In this device, biodiagnosis carried out for external stimuli of specific urea and uric acid is provided by synthetic genetic circuits that exhibit bio-recognition and bio-processing functions. In addition, sensors with capability of detecting multi-analytes simultaneously can be used for medical decision-making for complex diseases. Also, all these cell biosensors can be mounted to more complex hybrid devices as sensor interfaces and the result measurement methods can be adapted for a requested status. As a result, diagnosis of urea and uric acid concentrations is important for clinical, food and environmental industries. Therefore, an inexpensive, modular, user-friendly and quick urea/uric acid biodiagnosis device is a requirement with a wide application area.

Within these basic concepts; it is possible to develop various embodiments of the inventive medical diagnostic device; the invention cannot be limited to examples disclosed herein and it is essentially according to claims.

1-36. (canceled)

37. A medical diagnostic device characterized in that it comprises cellular biosensors performing diagnosis of urea and uric acid; and it is used for diagnosis of various diseases such as routine blood analysis and monitoring biomarkers about kidney health in the medical field.

38. A medical diagnostic device according to claim 37, characterized in that it has capability of performing low-cost, quick scanning and characteristic of providing high-specificity and yield, by means of cellular sensors it contains

39. A medical diagnostic device according to claim 37, characterized in that it comprises cellular biosensors that can perform urea detection, uric acid detection, urea and uric acid detection separately, and can detect that urea and uric acid are present in one environment at the same time.

40. A medical diagnostic device according to claim 37, characterized in that biosensors comprise synthetic genetic circuits.

41. A medical diagnostic device according to claim 37, characterized in that it has synthetic genetic circuits which are essentially configured to have transcriptional regulator and bio-sensing module that vary by the component aimed to be detected.

42. A medical diagnostic device according to claim 37, characterized in that it ensures formation of fluorescent signal increase in case of detecting urea and/or uric acid in the environment, and enables to detect this signal increase by fluorescence spectroscopy

43. A medical diagnostic device according to claim 37, characterized in that the uric acid (X) used for detecting uric acid is cloned from uricase operator system in transcriptional regulator HucR (C) and its DNA binding site HucO (E), organism Deinococcus radiodurans for cellular biosensor parts.

44. A medical diagnostic device according to claim 37, characterized in that HucR (C) is generated by promotor proD (A) continuously.

45. A medical diagnostic device according to claim 37, characterized in that synthetic promoter synpHucO (E) consists of HucO (E) operator placed between −35, −10 regions of viral promoter pL.

46. A medical diagnostic device according to claim 37, characterized in that synthetic promoter synpHucO (E) is activated depending on the presence of uric acid because it contains HucO binding site.

47. A medical diagnostic device according to claim 37, characterized in that entry of uric acid into the cell is possible by uric acid transporter (UACT) (H) gene cloned between minimal promotor mproD (G) that is continuously active on low copy plasmid pZS and RBS site (B) and T7 terminator (D) site.

48. A medical diagnostic device according to claim 37, characterized in that the urea (Y) used for diagnosing urea is cloned from transcriptional regulator UreR (J) from cellular biosensor parts and urease specific promoter region (Intergenic Region) (K) used as a promoter that is inducible by urea, urease operon system of Proteus mirabilis organism.

49. A medical diagnostic device according to claim 37, characterized in that a system consisting of all components of independent urea and uric acid detection and processing modules for a cellular biosensor with separate reporter of urea and uric acid—that is used for diagnosing urea (Y) and uric acid (X)—is designed.

50. A medical diagnostic device according to claim 37, characterized in that a synthetic promoter which is active only in the presence of urea and uric acid, is used in the urea and uric acid biosensor with AND-Logic gate used for diagnosing that urea (Y) and uric acid (X) are present in the environment at the same time.

51. A medical diagnostic device according to claim 37, characterized in that cloning a HucO (E) binding site between −35 and −10 regions of a promoter on the urease specific promoter region (K) makes the transcription activation dependent on the presence of both urea and uric acid.
